(19) United States
(12) Patent Application Publication
(10) Pub. No.: US 2021/0355160 A1
(43) Pub. Date: Nov. 18, 2021

(54) PROCESS FOR THE PURIFICATION OF LIPOPOLYPEPTIDE ANTIBIOTICS

(71) Applicant: GNOSIS S.P.A., Milano (IT)

(72) Inventors: Ermanno Valoti, Dalmine (IT); Mauro Rossini, Desio (IT); Roberto Auro Tagliani, Desio (IT)

(73) Assignee: GNOSIS S.P.A., Milano (IT)

(21) Appl. No.: 16/467,832

(22) PCT Filed: Dec. 12, 2017
(86) PCT No.: PCT/EP2017/082408

§ 371 Date: Jun. 7, 2019

(30) Foreign Application Priority Data
Dec. 16, 2016 (IT) 102016000127655

(51) Int. Cl. C07K 1/36; C07K 1/20; C07K 1/34; C07K 1/18; C07K 7/64

(52) U.S. Cl. CPC C07K 1/36; C07K 1/20; C07K 7/64; C07K 1/18; C07K 1/34

(57) ABSTRACT

Disclosed is a process for the purification of lipopolypeptide antibiotics from culture broths which comprises: a) removal of the mycelium from the broth; b) anion-exchange chromatography of the solution resulting from stage a), eluting with di- or trivalent ions; c) optional concentration of the purified fraction resulting from stage b); d) hydrophobic interaction chromatography of the fraction resulting from stage b) or c), eluting with C1-C4 alcohols; e) cation-exchange chromatography of the desired lipopolypeptide-enriched fraction resulting from stage d), eluting at a pH equal to or greater than the isoelectric point of the lipopolypeptide; and f) dialysis, concentration and freeze-drying or spray-drying of the purified lipopolypeptide.

### TECHNICAL FIELD

The invention relates to a process for the purification of lipopolypeptide antibiotics, in particular daptomycin and surotomycin, using ion-exchange chromatographies combined with adsorption chromatography.

### BACKGROUND

Daptomycin is an antibiotic used to treat antibiotic-resistant infections.

In 1978 (U.S. Pat. No. 4,208,403 and Re32,333), Eli Lilly researchers discovered antibiotic activity in the culture broths of Streptomyces roseosporus, which produces a mixture of polypeptides called “complex A-21978”; this mixture was separated into various fractions, one of which, namely fraction A-21978C, is particularly interesting. Said fraction comprises various compounds or factors which differ in terms of the fatty-acid chain bonded to the polypeptide; the C0 factor is a minor factor, and leads to different isomers with a decanoyl chain (U.S. Pat. No. 4,537,717).

The polypeptide nucleus common to the factors of A-21978C was subsequently obtained by biotransformation, and various derivatives were prepared by synthesis from that nucleus, including the decanoyl-derivative, initially called LY146032. As the decanoyl-derivative (obtained pure by semisynthesis, but also one of the isomers present in factor A-21978C0 obtained by fermentation) presents the best ratio between toxicity and efficacy, it was selected for clinical trials with the name of daptomycin (INN).

For industrial production, however, it is more economical to obtain the antibiotic by fermentation, administering the precursor decanoic acid, as described in U.S. Pat. No. 4,885,243. Although it is toxic for the micro-organism, decanoic acid enables a good quantity of daptomycin to be obtained.

A-21978C10, LY146032 and daptomycin are equivalent names corresponding to the active ingredient used in treatment, while the code A-21978C0 indicates a mixture of isomers having the same polypeptide nucleus but various alkyl chains (including n-decanoyl), and does not correspond to the pharmaceutical product.

Surotomycin (EP2379580B1) is a lipopolypeptide antibiotic particularly useful in Clostridium difficile infections. It is obtained by semisynthesis from daptomycin, after removing the alkyl chain (decanoic acid) of daptomycin and replacing it with an arylalkyl chain; the two products therefore have the same polypeptide structure in common, and only differ in terms of the lipid chain.

The aqueous solution of daptomycin produced by fermentation of Streptomyces roseosporus, administering decanoic acid (EP0178152, EP1586580) or analogues thereof (U.S. Pat. No. 4,885,157, EP2149609), presents high contamination by impurities, both correlated to daptomycin (polypeptides) and aspecific (mineral salts, sugars, proteins, etc.). The list of the main chemically correlated impurities is reported in EP01252179: about 15 of the impurities identified are polypeptides, some of which are biosynthesis intermediates while some derive from parallel biosynthesis or are daptomycin degradation products: Kirsch et al., Pharmaceutical Research 6, 5, 387-393 (1989).

Conversely, the purity requirements of the pharmaceutical product are very high: unlike other antibiotics (e.g. teicoplanins, wherein the medicament consists of a family of structurally similar products), in the case of daptomycin the correlated substances are not considered useful for treatment, but considered as unwanted impurities; the commercial product must have a purity exceeding 90% as daptomycin. U.S. Pat. No. 5,912,226 describes some of the main impurities correlated with daptomycin, such as the anhydrous form and the beta-isomer, and describes as the “substantially pure form” a daptomycin preparation which has less than 2.5% of said two impurities combined.

The manufacture of the active ingredient must therefore follow an elaborate purification process to obtain a pharmaceutical-grade product.

The purification of the antibiotic from the fermentation broths is hindered by the fact that while the aspecific impurities are easy to eliminate, other impurities consist of substances very similar to daptomycin, which cannot be easily separated with the simple, inexpensive techniques traditionally used in this field, such as extraction with solvent and ultrafiltration. Moreover, crude daptomycin cannot be purified by crystallisation. Although precipitation of the pure product from aqueous solutions has been described (EP1908770), this technique is not applicable to solutions of the crude product, as in that case, precipitation can give rise to the product in solid form, but without a significant increase in purity.

The situation is further complicated by the fact that the stability of the product in aqueous solution is not high, and spontaneous degradation readily occurs even at a neutral pH (and is even worse at acidic or alkaline pH), with the formation of three main compounds called beta-isomer, anhydrous-daptomycin and lactone-hydrolysis (Kirsch et al., Pharmaceutical Research 6, 5, 387-393, 1989), which are structurally very similar to daptomycin, and therefore difficult to separate. The first degradation products can obviously degrade in turn, giving rise to other impurities.

Daptomycin manufacturing processes are therefore based on purification by chromatography, with various steps on ion-exchange and/or adsorbent resins, until a product of high chemical purity is obtained.

The key step in all the processes known to date consists of reverse-phase or hydrophobic interaction chromatography, similar techniques which can be conducted on fixed phases based on derivatized silica (RP) with lipophilic chains (typically C8-C18 alkyl or phenyl chains) or on adsorbent resins (HIC), with lipophilic chains which are similar or devoid of functional groups. Due to the prohibitive cost of derivatized silica, the RP technique is only suitable for use on a laboratory scale, while adsorbent chromatographic resins are mainly used on an industrial scale, despite their lower separation efficiency. As the resins are also very expensive but easily soiled, gradually losing their separation capacity, it is generally preferable to pre-treat the fermentation broths by another technique, applying HIC downstream.

As a single chromatographic step is generally insufficient to achieve a satisfactory degree of purity, the chromatography must be repeated under the same conditions (US RE390,071), or a second purification step conducted on the same resins but at a different pH (US RE390,071, EP2398817), or a different purification technique added, such as ion-exchange chromatography (U.S. Pat. No. 6,696,412). The product can also be purified by reverse-phase chromatography (RP), using reverse phases in derivatized silica (U.S. Pat. No. 4,331,594, example 4), but the technique is not economical due to the cost of the phases in derivatized silica, the cost of the equipment (preparative HPLC) required to work at high pressures, and the high solvent consumption.

Both techniques require the use of water-miscible organic solvents, which must then be separated from the product. The use of solvents also involves safety problems due to their inflammability, and risks to the health of workers exposed to the vapors; the solvents must also be recovered or disposed of, which involves obvious ecological and financial drawbacks.

Ion-exchange chromatography (IEC) is based on the bond between the product and a positively- or negatively-charged fixed phase, while detachment is generally obtained with buffers having high ionic strength. In the case of daptomycin, which is highly unstable at alkaline pHs, resins with basic functionalisation are used (a weak base, such as diethylaminoethyl, or a strong base, such as quaternary ammonium), and the process is conducted at a neutral or weakly acid pH, loading the solution to be purified at low ionic strength and then eluting the product with solutions of gradually increasing ionic strength, typically obtained by increasing concentrations of sodium chloride. In the specific case of daptomycin, resins with diethylaminoethyl functionalisation (weak base) and an acrylic or methacrylic matrix with controlled porosity, which are suitable for use with macromolecules such as proteins and nucleic acids, have been used to date; this allows the product to be detached from the resin under conditions which are not too drastic, thus preventing degradation of the product. In particular, resins such as Diaion FPDA13, Amberlite IRA68 or other resins can be used.

In combination with purifications by chromatography, cheaper techniques such as extraction in organic solvent (immiscible with water) can also be used; n-butanol or n-butyl acetate is typically used (U.S. Pat. No. 4,331,594, WO2009/144739) to extract the product from solutions with a strongly acid pH (below the pI of daptomycin). The phases are separated, eliminating the aqueous phase, and the organic phase is then extracted with an aqueous solution buffered to a neutral pH, containing the product in dissociated form. This is an inexpensive process, which eliminates some aspecific impurities but does not guarantee any purification from structurally correlated impurities.

Tangential filtration techniques are also used to separate the mycelium (microfiltration), concentrate and/or dialyze the solutions, eliminate the solvent (ultra- and nanofiltration, reverse osmosis) or remove pyrogens (ultrafiltration).

As described in U.S. Pat. No. 4,331,594, the product can be purified by reverse-phase chromatography which, however, is not widely used on an industrial scale due to the cost of RP-18 silica, the cost of the necessary equipment, and the excessive consumption of organic solvent. Purification on RP-18 phases was therefore considered unsatisfactory for industrial application, and cheaper alternatives were sought to produce the antibiotic at a reasonable cost.

The method described in U.S. Pat. No. 4,874,843 involves separation by filtration of the biomass at the end of fermentation from the liquid phase containing the product, and absorption of daptomycin on Diaion HP20 adsorbent resin. After elution, the semipure daptomycin is purified by a succession of steps on Diaion HP20 and Diaion HP20ss, a better-quality version of the same resin, suitable for HIC. However, a single step is not sufficient; the solvent must be removed and the daptomycin solution must then undergo at least one more chromatographic step. The purity of the resulting product is not high, and the process requires the use of large amounts of solvent.

The method described in U.S. Pat. No. 6,696,412, which is commercially feasible to produce daptomycin with a high degree of purity, consists of a series of successive chromatographic operations comprising anion-exchange chromatography, hydrophobic interaction chromatography (HIC) and a second anion-exchange step. The stated purity of the resulting daptomycin exceeds 95%. A particularly economical application of said process involves separating the mycelium from the aqueous solution at the end of fermentation, using a special apparatus called the PallSep, to obtain a clear solution of daptomycin. Said solution is fluxed on an anionic resin, which retains daptomycin but does not retain most of the aspecific impurities present in the culture medium; the desired product is eluted from the resin with a sodium chloride solution at increasing concentrations, to obtain a saline aqueous solution of daptomycin, with increased purity. According to the patent teachings, Mitsubishi Diaion FPDA13, an acrylic resin with DEAE (diethylaminoethyl) functionalisation, is particularly suitable. The resulting solution then undergoes concentration and/or dialysis by ultrafiltration, a step wherein most of the salt present in the elution buffer is eliminated in the permeate, while the daptomycin is concentrated in the retentate; in a variation on the process, the ultrafiltration phase can be replaced by simple dilution with water.

The partly purified, desalted, concentrated solution is then loaded onto a chromatography column packed with an adsorbent resin, Diaion HP20ss, and eluted with increasing concentrations of a water-miscible organic solvent (acetonitrile, isopropanol or the like); the required purity of the end product is reached in this step, while the subsequent phases do not involve substantial purification of the product but actually risk reducing its purity, due to the spontaneous degradation of daptomycin. As pure daptomycin solutions contain high percentages of organic solvent, they are subjected to dilution and dialysis with water, by ultrafiltration, or can undergo further chromatography on FPDA13 resin, as in the process described above. Other ultrafiltration steps follow, to remove pyrogens and concentrate the solutions, which are finally freeze-dried to obtain the powdered product.

Micelles, namely aggregates of several molecules of daptomycin, form under the conditions described in this patent due to the particular characteristics of daptomycin, which comprises a lipid chain (decanoic acid) bonded to a polypeptide structure, and this phenomenon is expressly exploited in the ultrafiltration steps. The same patent also describes the use of chaotropic agents (e.g. urea) at high concentrations for the HIC purification phase. U.S. Pat. No. 8,058,238 and U.S. Pat. No. 8,129,342 give more details of the impurities present, and describe the analytical HPLC methods used to analyze the product.

Numerous purification methods involve a succession of different chromatography steps to obtain a product with adequate purity; for example, WO 2009/144739 describes the use of preparative RP-HPLC as the sole chromatography step to obtain daptomycin with purity levels exceeding 96%. The drawback of said approach is the high cost of the HPLC technique on a preparative scale, and the fact that it is unsuitable to prepare hundreds of kilos of the product.

Other patents, such as WO2009/144739 and EP2398817, report multi-stage purification schemes, based on the use of anionic resins and adsorbent resins optionally combined with extraction in organic solvent; both describe the use of sodium chloride or potassium chloride (or other alkaline or alkaline-earth halides) as being particularly advantageous.

As HIC chromatography conducted on resin is less efficient than RP on silica, it may be necessary to introduce new purification stages or, more simply, to repeat the chromatography at a different pH, consequently conducting one step at a neutral pH and one at an acid pH on the same resin (EP2398817).

The use of a cationic resin in the purification of daptomycin is described in CN101899094, wherein the purification is conducted by means of repeated steps with ultrafiltration and nanofiltration membranes with different cut-offs, due to the formation of micelles. The solution containing the product is then acidified and loaded, at a particularly low flow rate (0.3 volumes per hour), onto a sulfonic resin in acid form. The solution is eluted with an HCl gradient ranging from 0.01 N to 0.05 N, to obtain a product with 90% purity, which is then concentrated by nanofiltration and evaporation under vacuum, and finally crystallized. However, there are no indications of the yield, and the operating conditions, at an extremely acid pH for a long time, are incompatible with the stability of daptomycin (as reported in Kirsch et al., Pharmaceutical Research 6, 5, 387-393, 1989) and polypeptides in general. Moreover, the use of strongly acid HCl solutions leads to corrosion of the steel parts of the equipment, and consequently the presence of impurities made of iron, chromium and nickel ions, which are obviously undesirable in an injectable pharmaceutical product.

In all the literature known to date, daptomycin is eluted from an ion-exchange resin with high concentrations of sodium chloride, which is considered particularly suitable for that purpose, as described in U.S. Pat. No. 6,696,412 and EP2398817, which specifically claims the use of monovalent ions. The presence of sodium chloride seems to be particularly important in processes based on micelle formation, aided by the presence of salt and certain pH conditions, as reported in U.S. Pat. No. 8,129,342 (column 21).

Daptomycin is also known to have chelating properties; in particular, its action mechanism as an antibiotic is correlated with the formation of a bond with calcium ions (Shapiro et al., Antimicr Ag Chemother 47, 8 2538-44, 2003), but its chelating capacity can also be exploited in the purification process, for example by extracting the antibiotic-Ca complex in organic phase as described in EP1355920B1 (example 14, referring to the antibiotic mixture A-21978C). This is consequently not a chromatographic process but an alternative method of extracting the product in solvent; the degree of purification obtained is very low, and only aspecific impurities are eliminated, not the impurities most similar to daptomycin (which are more difficult to separate).

Moreover, the crystallisation of the pure product (EP1908770) does not involve the formation of complexes with Ca++ or other bivalent ions. In practice, in all the purification processes of the product known to date, apart from the above-mentioned extraction in solvent, the presence of bivalent ions in the daptomycin solutions tends to be avoided.

### SUMMARY OF THE INVENTION

The present invention describes a process for the purification of lipopolypeptide antibiotics, in particular daptomycin or surotomycin, characterized by eluent ion-exchange chromatography techniques based on bivalent or trivalent ion buffers, also at high concentrations, obtaining a good degree of purification. A cationic resin is also used, on which the product is loaded and eluted under pH conditions different from those known to date. In a variation on the process described, cation-exchange chromatography can conveniently be used to remove the solvent from lipopolypeptide antibiotic solutions. In a further variation, it can be used to decolorized the lipopolypeptide antibiotic solutions resulting from fermentation broths.

The process according to the invention comprises:

a) removal of the mycelium from the culture broth (by microfiltration, centrifugation or another process);

b) anion-exchange chromatography of the solution resulting from stage a), eluting with di- or trivalent ions;

c) optional concentration of the purified fraction resulting from stage b, in particular by nanofiltration;

d) hydrophobic interaction chromatography of the fraction resulting from stage b) or c), eluting with C1-C4 alcohols;

e) cation-exchange chromatography of the desired lipopolypeptide-enriched fraction resulting from stage d) by eluting with saline solutions, optionally at a pH higher than the isoelectric point of the lipopolypeptide;

f) dialysis, concentration and freeze-drying or spray-drying of the purified lipopolypeptide.

Stage b) is preferably eluted with magnesium sulphate, aluminium sulphate or a straight or cyclic diamine citrate, more preferably with magnesium sulphate.

The anion-exchange resin is preferably a resin functionalized with weak basic groups.

The elution of the hydrophobic interaction chromatography of stage d) is preferably conducted with isopropanol.

The cation-exchange chromatography of stage e) is conducted with a resin functionalized with strong acid groups, eluting at a pH ranging between 3 and 7.

The invention also relates to the use of a cation-exchange resin to remove water-miscible organic solvents from aqueous solutions of daptomycin or surotomycin and to decolorize solutions of daptomycin or surotomycin in water or in a mixture of water and water-miscible organic solvents.

### DETAILED DESCRIPTION OF THE INVENTION

Using the process according to the invention, bivalent or trivalent ions, which may be metal ions such as Mg, Zn and Al or bivalent, straight organic bases such as ethylenediamine, dimethylethylenediamine and the like, or cyclic bases such as imidazole, piperazine and the like, can be successfully used in ion-exchange chromatography for the purification of daptomycin. The salt can be formed with an inorganic or organic monovalent, bivalent or trivalent counterion, such as acetates, formates, tartrates, citrates, sulphates, chlorides, phosphates, and polyphosphates of bivalent organic bases or of bivalent ions of metallic or metalloid elements.

When the bivalent ion system is selected, account should be taken of the solubility of the salt in water, whether it is able to buffer the pH of the solution, and whether it is liable to give oxidation-reduction reactions in the presence of dissolved oxygen. Different saline systems can also be used at the various stages of the process; in particular, when conditioning the resin before use and regenerating it after use, buffer systems and saline systems different from those selected as eluent at the chromatography stage can be used.

The preferred saline system used as eluent is magnesium sulphate, employed at concentrations ranging from zero to 1 M, and in particular from zero to 600 mM, with a lower concentration at the start of chromatography which is then gradually increased until the highest value indicated is reached, with an incremental profile that can be either the step type (discontinuous) or the gradient type (continuous). Preferably, the magnesium sulphate can be combined with a buffer system used at a low concentration but still able to control the pH, maintaining it at the desired value. One example of a buffer system is magnesium acetate and acetic acid.

The same type of chromatography on anion-exchange resins can be obtained using buffers based on bivalent non-metallic ions, such as straight or cyclic diamines, also salified with a monovalent, bivalent or trivalent acid counterpart.

According to the invention, the use of bivalent ions as eluents for ion-exchange chromatography in the purification of daptomycin offers the advantages described below. The procedure is particularly useful to obtain good separation of some correlated impurities which are difficult to separate in known hydrophobic interaction chromatography processes, while simultaneously eliminating some aspecific impurities deriving from fermentation. In particular, the technique is directly applicable to fermentation broths, preferably after separating the mycelium by centrifugation or microfiltration, a good degree of purity already being obtained after the first chromatography step.

Various aqueous solutions can be used for this purpose, comprising a) a buffer system, which can be of any type, organic or inorganic, provided that it can buffer at a pH ranging from 2 to 7, and b) a bivalent salt, which is used at increasing concentrations and has the task of selectively causing the daptomycin and the correlated impurities to detach from the resin, which are divided into different fractions. A variation on the process described herein involves the use of the same salt to control both the pH and the ionic strength, for example by using it at a low concentration for pH control only, and then increasing the concentration to obtain the elution of the product.

Various types of ion-exchange resins can be used for this purpose, based on natural polymers like dextran and agarose, and on synthetic polymers like polymethacrylates and polystyrenes; weak bases like diethylamines and strong bases like quaternary ammonium ions can both be used as functional groups. Polymethacrylic resins with a diethylaminoethyl function, such as Diaion FPDA13 resin, are particularly suitable for this purpose, due to their low cost and the absence of aspecific interactions; said resins bond to daptomycin in the pH range wherein the product is most stable, and then release it with good yields.

Unlike calcium, some bivalent ions do not interfere with the process of bonding to the resins, so that the fractions obtained by purification with anionic resin can be used directly in HIC chromatography, with no need for dialysis or concentration steps.

A second field of application of chromatography on ion-exchange resin using bivalent ions is desolvation of daptomycin solutions, such as the fractions obtained by HIC chromatography. As already stated, HIC chromatography uses increasing quantities of water-miscible organic solvents to elute the product adsorbed on the resin; acetonitrile, isopropanol, ethanol or other similar solvents can be used, at variable concentrations

The invention is illustrated in greater detail in the examples below.

“Purity” here means the percentage ratio between the peak area of daptomycin and the sum total of the peak areas of daptomycin and the impurities, determined by HPLC analysis with a UV detector at 214 nm, as described in U.S. Pat. No. 8,129,342 (column 22). Where indicated, the individual impurity contents relate to the ratio between the peak area of the substance indicated and the total of the areas, determined by HPLC as above.

### EXAMPLE 1

A culture of Streptomyces roseosporus is grown in submerged aerobic fermentation as described in patent EP0178152B1, administering decanoic acid during the final stages of fermentation and taking the necessary precautions to prevent its accumulation, as described in patent U.S. Pat. No. 4,208,403.

40 liters of a suspension containing about 2.5 grams of daptomycin per gram of fermentation broth is obtained, and purified in the following steps:

a) The whole broth undergoes microfiltration using titanium dioxide-based membranes with suitable porosity (0.2 μm). An almost clear filtrate is obtained, and conveyed to the subsequent nanofiltration stages; the mycelium in the retentate is resuspended in water and microfiltered again, and the second filtrate is combined with the first to improve recovery of the product. Finally, a dark aqueous solution is obtained, with a daptomycin concentration of about 1.5 g/l, corresponding to a process yield of over 90%. The solution is partly concentrated by nanofiltration, eliminating the permeate (which is devoid of product) and retaining the retentate; to shorten the processing time and limit degradation of the product, nanofiltration is conducted at low temperature, and commenced simultaneously with microfiltration. A reddish-brown concentrated solution of crude daptomycin is obtained, with a purity of about 50-55% in the HPLC area (determined as described above), which is called the microfiltered broth;

b) The microfiltered broth is loaded, corrected to pH=6, and loaded onto a Diaion FPDA13 anionic resin column, pre-balanced with a buffer solution of 50 mM magnesium acetate at pH 6. The daptomycin bonds entirely to the resin, while a clear, colored solution is eliminated in the effluent. The resin is washed with demineralized water, then with a buffer solution of 50 mM magnesium acetate at pH=6; the effluent obtained from the column mainly contains impurities, and is eliminated. The product is eluted from the resin with a solution of 50 mM magnesium acetate and magnesium sulphate ranging from zero to 500 mM at pH 6, dividing the effluent into various fractions, followed by HPLC analysis of each fraction as described above. The fractions with adequate purity are combined, then concentrated by nanofiltration, using polymer membranes with a cut-off of about 500 Da; no micelle formation is observed;

b) The daptomycin solution is loaded onto a Diaion HP20ss resin column, pre-conditioned in 50 mM ammonium acetate buffer at a pH of about 6.3, and packed under pressure in a fixed-bed container. The solution leaving the column during loading is discarded, and a volume of demineralized water equal to the volume of resin is loaded, discarding the leaving solution. The product is eluted with a 50 mM pH 6 ammonium acetate buffer solution with increasing quantities of isopropanol, increasing the solvent concentration in a gradual linear progression from 5% to 40% (by volume); the leaving solution is fractionated in portions amounting to half the volume of resin. The fractions are analysed by HPLC and combined or discarded on the basis of the daptomycin purity data in area % by the method indicated above;

d) The purified solution of daptomycin is diluted with an equal volume of demineralized water, then loaded onto a Relisorb SP400 (Resindion) resin column pre-conditioned to pH 3 with dilute formic acid. The resin is washed with an 0.1% formic acid solution diluted in water for injection (WFI), using two volumes of solution per volume of resin; at this stage, the loss of product in the effluent is almost nil. The daptomycin is eluted from the resin with an aqueous solution of 100 mM ammonium acetate at pH 5, then concentrated by nanofiltration until the volume is reduced to ⅕th of the initial volume. The concentrate is dialyzed with WFI, adding it continuously to the retentate in quantities equal to the permeate flow.

The resulting daptomycin solution is further concentrated until a concentration of 130 g/l is reached, and then freeze-dried. Powdered daptomycin with 96% purity and a residual magnesium content of less than 10 ppm is obtained.

### EXAMPLE 2

The fermentation and microfiltration of S. roseosporus are conducted as described in example 1:

a) Microfiltered broth 1 is corrected to pH=6 and loaded onto a column of Diaion FPDA13 anionic resin, pre-balanced with a buffer solution of 50 mM magnesium acetate at pH 6; the daptomycin bonds entirely to the resin, while a clear, colored solution is eliminated in the effluent. The resin is washed with demineralized water, then with a buffer solution of 50 mM magnesium acetate at pH=6; the effluent obtained from the column mainly contains impurities, and is eliminated. The product is eluted from the resin with a solution of 50 mM magnesium acetate and aluminium sulphate ranging from zero to 300 mM at pH 6, dividing the effluent into various fractions. HPLC analysis of each fraction is then conducted as described above; the fractions with adequate purity are combined and concentrated by nanofiltration, without observing micelle formation;

b) The partly purified solution is loaded onto a column of Purolite PCG1200M resin, pre-conditioned in 50 mM ammonium acetate buffer at a pH of about 6.3, and packed under pressure in a fixed-bed container. The solution leaving the column during loading is discarded, and a volume of demineralized water equal to the volume of resin is loaded, discarding the leaving solution. The product is eluted with a 50 mM pH 6 ammonium acetate buffer solution with increasing quantities of ethanol, increasing the solvent concentration in a gradual linear progression from 10% to 60% (by volume); the leaving solution is fractionated in portions amounting to half the volume of resin. The fractions are analysed by HPLC and combined or discarded on the basis of the daptomycin purity data in area % by the method indicated above. An aqueous solution containing ethanol is obtained, wherein daptomycin is present with a purity of about 96%;

c) The purified solution of daptomycin is diluted with an equal volume of demineralized water, then loaded onto a Relisorb SP400 (Resindion) resin column pre-conditioned to pH 3 with dilute formic acid. The resin is washed with an 0.1% formic acid solution diluted in water for injection (WFI), using two volumes of solution per volume of resin; at this stage, the loss of product in the effluent is almost nil. The daptomycin is eluted from the resin with an aqueous solution of 500 mM magnesium sulphate at pH 3, then concentrated by nanofiltration, dialyzed and freeze-dried as described in example 1.

Powdered daptomycin with a purity exceeding 95% is obtained.

### EXAMPLE 3

a) The microfiltered broth obtained as described in example 1 is corrected to pH 6.0-6.5 with acetic acid and loaded onto a Diaion FPDA13 anionic resin column, pre-balanced with a buffer solution of 50 mM piperazine citrate at pH 6; the daptomycin bonds entirely to the resin, while a clear, colored solution is eliminated in the effluent. The resin is washed with demineralized water, and then with a buffer solution of 50 mM piperazine citrate at pH 6; the effluent obtained from the column mainly contains impurities, and is eliminated;

The product is eluted from the resin with a solution of piperazine citrate ranging from 50 mM to 200 mM at pH 6, dividing the effluent into various fractions, followed by HPLC analysis of each fraction as described above; the fractions with adequate purity are combined and concentrated by nanofiltration, without observing micelle formation;

b) The solution of the concentrated product is acidified to pH 3.8, and then subjected to liquid/liquid extraction, adding an equal volume of n-butanol; the daptomycin is again extracted from the butanol solution with a small volume (½ the solvent volume) of aqueous buffer at pH 6.3. The solution is distilled under vacuum to reduce the residual quantity of solvent;

c) The solution is loaded onto HP20ss resin as described in example 1, paragraph c), but using solutions with an increasing isopropanol concentration. The fractions with purity exceeding 95% in HPLC area are selected and combined;

d) The purified solution of daptomycin is diluted with an equal volume of demineralized water, then loaded onto a Relisorb SP400 (Resindion) resin column pre-conditioned to pH 3 with dilute formic acid. The resin is washed with an 0.1% formic acid solution diluted in water for injection (WFI), using two volumes of solution per volume of resin; at this stage, the loss of product in the effluent is almost nil. The daptomycin is eluted from the resin with an aqueous solution of 500 mM sodium chloride in 20% ethanol at pH 3, then concentrated by nanofiltration, dialyzed and freeze-dried as described in example 1.

### EXAMPLE 4

a) The microfiltered broth obtained as described in example 1 is corrected to pH 6.0-6.5 and loaded onto a Diaion FPDA13 anionic resin column, pre-balanced with a buffer solution of 50 mM ethylenediamine acetate at pH 6; the daptomycin bonds entirely to the resin, while a clear, colored solution is eliminated in the effluent. The resin is washed with demineralized water, and then with a buffer solution of 50 mM ethylenediamine acetate at pH 6; the effluent obtained from the column mainly contains impurities, and is eliminated. The product is eluted from the resin with a solution of 50 mM to 300 mM ethylenediamine acetate at pH 6, dividing the effluent into various fractions, followed by HPLC analysis of each fraction as described above; the fractions with adequate purity are combined;

b) The resulting daptomycin solution is adjusted to pH 3 with hydrochloric acid, then further purified with Purolite PCG1200M resin. The product is eluted with a 50 mM pH 6 ammonium acetate buffer solution with increasing quantities of isopropanol, increasing the solvent concentration in a gradual linear progression from zero to 40% (by volume). The fractions are analysed by HPLC and combined on the basis of purity;

c) The pure daptomycin solution is desolvated, correcting to pH 3 and capturing the product on Relite SP400 resin. The resulting product is eluted with a sodium acetate solution at pH 6, obtaining a quantitative yield. The solution is then dialyzed with water by nanofiltration on polysulphone membranes with a cut-off of 500 Da, concentrated to 100 g/l and freeze-dried.

### EXAMPLE 5

a) The fermentation and microfiltration are conducted as described in example 1, with the difference that the microfiltered broth is loaded directly onto the FPDA13 resin pre-conditioned with acetate buffer at pH 6. Demineralized water equal to two volumes of resin is loaded, then eluted with an ammonium acetate buffer containing ammonium sulphate in increasing quantities from 50 mM to 500 mM, at pH 6;

b) The resulting solution is loaded directly (at the same concentration) onto a Purolite PCG1200M resin column, pre-conditioned with formic acid at pH 3 and packed under pressure in a fixed-bed container. The solution leaving the column during loading is discarded, and a volume of demineralized water equal to the volume of resin is loaded, discarding the leaving solution. The product is eluted with a solution containing increasing quantities of isopropanol with the addition of formic acid to pH 3, increasing the solvent concentration in a gradual linear progression from zero to 50% (by volume); the leaving solution is fractionated in portions amounting to half the volume of resin. The fractions are analysed by HPLC and combined on the basis of the daptomycin purity data;

c) The pure daptomycin solution is desolvated with Relite SP400, loading at pH 3 and eluting with ammonium acetate buffer at pH 7. The yield obtained is quantitative.

### EXAMPLE 6

a) The microfiltered solution obtained in example 1 is loaded onto a column containing Amberlite 1200H cationic resin pre-balanced with 50 mM sodium acetate buffer at pH 6; a yellow solution containing about the same concentration of daptomycin leaves the column. The resin is further eluted with the same buffer, using a quantity by volume equal to twice the volume of resin; the solutions eluted are concentrated by ultrafiltration without observing micelle formation. A bleached solution with a 95% daptomycin yield is obtained;

b) The solution is concentrated by nanofiltration, then acidified to pH 3 with HCl and extracted with an equal volume of n-butanol; after separation, the aqueous phase is discarded. The organic phase is extracted with a 50 mM buffer solution of ammonium acetate, adding ammonia to correct the pH to 6;

c) The resulting solution is purified by chromatography on Relite Diaion HP20 resin, eluting with a linear gradient of ethanol from zero to 60%, at pH 6. The fractions with a purity exceeding 85% are selected, and desolvated as described in example 5, point c). The solution is dialyzed and concentrated by nanofiltration on 500 Da membranes.

### EXAMPLE 7

The purification of the microfiltered broth proceeds as described in example 1, up to point c), with the difference that the desolvation is conducted with anionic resin. The aqueous solution of daptomycin originating from HIC chromatography, containing isopropanol, is loaded onto an FPDA13 resin column pre-conditioned to pH 6 with magnesium acetate buffer.

The resin is washed with water, used in quantities equal to twice the volume of resin. The product is eluted with a solution of 500 mM magnesium sulphate and 50 mM magnesium acetate at pH 6.

The resulting solution is concentrated with a nanofilter and dialyzed with water; the solution is corrected with HCL to pH 3, and finally, further concentrated to 130 g/l. The solution is frozen and freeze-dried under high vacuum, to obtain a pale yellow powder consisting of daptomycin with 96% purity containing less than 10 ppm of magnesium.

1. A process for the purification of lipopolypeptide antibiotics from culture broths which comprises:
a) removal of the mycelium from the broth to provide a solution;
b) anion exchange chromatography of the solution from step a) eluting with di- or tri-valent ions to provide a purified fraction;
c) optional concentration of the purified fraction from step b);
d) hydrophobic interaction chromatography of the purified fraction from step b) or c) eluting with C1-C4 alcohols to provide a lipopolypeptide-enriched fraction;
e) cation exchange chromatography of the lipopolypeptide-enriched fraction from step d) eluting at a pH equal to or higher than the lipopolypeptide isoelectric point to provide a purified lipopolypeptide antibiotic; and;
f) dialysis, concentration and freeze-drying or spray-drying of the purified lipopolypeptide antibiotic.

2. The process according to claim 1 wherein the lipopolypeptide antibiotic is daptomycin or surotomycin.

3. The process according to claim 1 wherein elution of step b) is carried out with magnesium sulfate, of aluminum sulfate or a straight or cyclic diamine citrate.

4. The process according to claim 3 wherein elution of step b) is carried out with magnesium sulfate.

5. The process according to claim 1 wherein step b) is carried out using a resin functionalized with weak basic groups.

6. The process according to claim 1 wherein elution of hydrophobic interaction chromatography of step d) is carried out with isopropanol.

7. The process according to claim 1 wherein the cation exchange chromatography of step e) is carried out using a resin functionalized with strong acid groups.

8. The process according to claim 7 wherein the resin is eluted at pH ranging from 3 to 7.

9. The process according to claim 1 wherein a cation exchange resin is employed for removing water-miscible organic solvents from daptomycin or surotomycin aqueous solutions.

10. The process according to claim 1 wherein a cation exchange resin is employed for decolorizing daptomycin or surotomycin in water or water-miscible organic solvents solutions.
